(19) United States
(12) Patent Application Publication
(10) Pub. No.: US 2022/0371009 A1
(43) Pub. Date: Nov. 24, 2022

(54) SYSTEM AND METHOD FOR SELECTIVE MICROCAPSULE EXTRACTION

(71) Applicant: University of Maryland, College Park, College Park, MD (US)

(72) Inventors: Xiaoming He, Bethesda, MD (US); Alisa White, Greenbelt, MD (US)

(21) Appl. No.: 17/725,096

(22) Filed: Apr. 20, 2022

Related U.S. Application Data
(60) Provisional application No. 63/177,297, filed on Apr. 20, 2021.

(51) Int. Cl. B01L 3/00; G06V 20/69

(52) U.S. Cl. CPC B01L 3/50273; B01L 3/502715; G06V 20/69

(57) ABSTRACT

A system for selective microcapsule extraction includes a non-planar core-shell microfluidic device. The non-planar core-shell microfluidic device generates microcapsules defining a core-shell configuration. A subset of the microcapsules contain aggregates, tissues, or at least one cell. A camera captures images of the microcapsules. A detection module includes a processor and a memory. The memory includes instructions that when executed by the processor causes the detection module to provide the images of the microcapsules as an input to a machine learning model. The machine learning model identifies microcapsules containing aggregates, tissues, or at least one cell. A force generator generates a force to extract the microcapsules. A microcontroller selectively activates the force generator to generate the force when the detection module identifies a microcapsule containing aggregates, tissues, or at least one cell to extract the microcapsule.

### CROSS-REFERENCE TO RELATED APPLICATION

The present application claims priority to U.S. Provisional Patent Application No. 63/177,297 filed on Apr. 20, 2021, the entire contents of which are incorporated by reference herein.

### GOVERNMENT SUPPORT

This invention was made with government support under R01EB023632B awarded by the National Institutes of Health. The government has certain rights in the invention.

### FIELD

The present disclosure relates to microcapsule extraction and, more particularly, to a system and method for selective microcapsule extraction.

### BACKGROUND

Microencapsulation of cells and tissues has become a very popular area of research for its ability to encase cells in a 3D environment which can be utilized in cell-based medicine including tissue engineering, regenerative medicine, and cell-based therapies. For example, microcapsules can be used to create a biomimetic environment for ovarian follicles and a biocompatible immunogenic barrier for stem cell and islet transplantation. Microfluidics has been widely explored for cell and tissue encapsulation because of its good controllability in terms of the size, morphology, and composition of the microcapsules. Microfluidic cell encapsulation is usually accomplished by shearing cell-laden aqueous fluids with a water immiscible fluid (e.g., oil). One major challenge is that the percentage of cell-laden microcapsules to total microcapsules generated is very small (<5%) due to the low cell density required for encapsulating only one piece of cell/tissue or cell aggregate per microcapsule, and in the case of pancreatic islets and ovarian follicles, the limited amount of these precious tissues that can be isolated. This large number of empty microcapsules makes downstream processing difficult, and in particular, undesired for cell/tissue transplantation for which the space available in vivo for housing the transplants is very limited. To remove empty microcapsules, sorting is needed. This can be done manually or by moving all the microcapsules to another device for sorting, which is tedious and may cause sample loss, contamination, and/or, cell damage. Another challenge is that oil is not favorable for cell viability. Therefore, an effective method for selective extraction of cell-laden microcapsules from oil into an isotonic aqueous phase timely on-chip can be very important to maintain the cell quality for use in clinical settings.

Several methods have been reported for extraction of microcapsules from oil into an aqueous phase based on the size and/or natural hydrophilicity of the microcapsule surface. However, these studies have not explored selective extraction of cell-laden microcapsules. Several studies reported optical detection of cell-laden microcapsules to selectively extract them. However, these methods require either fluorescently labeling the cells or squeezing the cell-laden constructs. The former may negate the utility of the cell-laden microcapsules for cell-based medicine while the latter may damage the cell-laden hydrogel microcapsules permanently. More recently, a label-free method was demonstrated for on-chip selective extraction of cell aggregate-laden microcapsules from oil into an aqueous solution using an optical sensor and dielectrophoresis (DEP). However, the purity is low: only ˜30% of the extracted microcapsules are cell-laden. In addition, cell aggregates less than ˜80 μm in diameter are not detectable with the optical sensor-based method.

With the development of high-capacity graphical processing units (GPUs) and the availability of big data, deep learning has risen to prominence as an accessible tool for many fields in recent years, especially in the field of image processing. Deep learning is a type of machine learning that uses neural networks (nodes connected by varying weights) to make predictions based on the input data. Supervised learning is a type of deep learning in which a program can be used to classify different objects that are input into the system, by first “learning” from pre-labeled objects that are used to train the program. Object classification can be very useful for biomedical applications, and deep learning can be used to detect subtle features in images, which may surpass human performance. More recently, deep learning has been used to detect live and dead cells, as well as different types of cells. However, nearly all prior studies have been focused on analyzing static images offline. Two studies have used machine learning to identify and separate droplets containing cells from droplets without cells on microfluidic devices in real-time, but these studies require complex imaging setups with special photodetectors, do not result in high purity and efficiency in sorting, and/or do not create hydrogel microcapsules for further processing. One study has examined sorting hydrogel microcapsules (containing no cells/tissues) of different sizes by deep learning. The capability of deep learning in real-time detecting cell/tissue-laden hydrogel microcapsules for their label-free on-chip selective extraction has not been explored so far.

### SUMMARY

Provided in accordance with aspects of the present disclosure is a system for selective microcapsule extraction including a non-planar core-shell microfluidic device. The non-planar core-shell microfluidic device generates microcapsules defining a core-shell configuration. A non-planar microfluidic device contains channels of varying heights, which are employed to create a core-shell microcapsule. A subset of the microcapsules contain aggregates, tissues, or at least one cell. A camera captures images of the microcapsules. A detection module includes a processor and a memory. The memory includes instructions that when executed by the processor cause the detection module to provide the images of the microcapsules as an input to a machine learning model. The machine learning model identifies microcapsules containing aggregates, tissues, or at least one cell. A force generator generates a force to extract the microcapsules. A microcontroller selectively activates the force generator to generate the force when the detection module identifies a microcapsule containing aggregates, tissues, or at least one cell to extract the microcapsule.

In an aspect of the present disclosure, a microcapsule generated by the non-planar core-shell microfluidic device defines a biomimetic environment containing a plurality of single cells, a plurality of cell tissues, and a plurality of cell aggregates.

In an aspect of the present disclosure, the biomimetic environment includes at least one hydrogel surrounding the aggregates, tissues, or cell(s).

In an aspect of the present disclosure, the microfluidic device includes at least two immiscible phases. The force generator is selectively activated by the microcontroller to extract microcapsules from a first phase to a second phase.

In an aspect of the present disclosure, a core of a microcapsule generated by the non-planar core-shell microfluidic device is surrounded by an inner wall of a corresponding microcapsule.

In an aspect of the present disclosure, the core is centered in an inner space of the corresponding microcapsule.

In an aspect of the present disclosure, the machine learning model includes a machine learning classifier, or a convolutional neural network.

In an aspect of the present disclosure, the extracted microcapsule includes ovarian follicle cells, pancreatic islet cells, stem cells, or somatic cells. The ovarian follicle cells, pancreatic islet cells, stem cells, or somatic cells are arranged as a single cell, multiple cells, or cell aggregates.

In an aspect of the present disclosure, the non-invasive force generated by the force generator includes an electrical force or an acoustic force.

In an aspect of the present disclosure, the camera is a digital camera, a high speed digital camera, a camera embodied in a smartphone, or a camera embodied in a tablet computer.

Provided in accordance with aspects of the present disclosure is a computer-implemented method for selective microcapsule extraction including generating microcapsules. A subset of the microcapsules contain aggregates, tissues, or at least one cell. Images of the generated microcapsules are captured. The images are provided as an input to a machine learning model. The machine learning model identifies a microcapsule containing aggregates, tissues, or at least one cell based on the provided input. A non-invasive force is selectively applied to extract the microcapsule containing the aggregates, the tissues, or the at least one cell.

In an aspect of the present disclosure, the extracted microcapsule is provided as an in-vivo treatment, for an in-vitro study, or for cell analysis.

Provided in accordance with aspects of the present disclosure is a system for selective microcapsule or droplet extraction including a non-planar core-shell microfluidic device configured to generate microcapsules or droplets defining a core-shell configuration. A subset of the microcapsules or a subset of the droplets contain aggregates, tissues, or at least one cell. A camera captures images of the generated microcapsules or droplets. Images of the generated microcapsules or droplets are provided as an input to a machine learning model. The machine learning model identifies a microcapsule or droplet containing aggregates, tissues, or at least one cell. A force generator generates a force to be applied to the microcapsules or droplets. A microcontroller selectively activates the force generator to generate the force when the detection module identifies the microcapsule or the droplet containing the aggregates, tissues, or the at least one cell to extract the microcapsule or the droplet.

### BRIEF DESCRIPTION OF THE DRAWINGS

Various aspects and features of the present disclosure are described hereinbelow with reference to the drawings wherein:

FIG. 1 is a flow chart of a system for selective microcapsule extraction according to aspects of the present disclosure;

FIG. 2 is a schematic diagram of the system of FIG. 1;

FIG. 3 is a schematic diagram of exemplary separation phases employable by the microfluidic device of the system of FIG. 1;

FIG. 4. is an enlarged view of the steps performed by the microfluidic device of FIG. 3;

FIG. 5 is a schematic diagram of exemplary separation phases employable by the microfluidic device of the system of FIG. 1;

FIG. 6A illustrates a comparison between an alginate microcapsule and a core-shell microcapsule;

FIG. 6B illustrates a comparison between an islet cell encapsulated in an alginate microcapsule and an islet cell encapsulated in a core-shell microcapsule;

FIG. 7A is an illustration of an exemplary deep learning detection model including a backend architecture and a convolutional neural network using a single shot multibox detection (SSD), for detecting cell aggregate-laden microcapsules;

FIG. 7B is an exemplary graph showing image acquisition speeds captured by a camera of the system of FIG. 1;

FIG. 7C is an exemplary graph of deep learning detection models with three different backend structures;

FIG. 8A illustrates exemplary images of the selective extraction of a cell aggregate-laden microcapsule from oil into an aqueous extraction solution;

FIG. 8B illustrates a comparison between microcapsule extraction without selective extraction and with selective extraction;

FIG. 8C is a graph of exemplary data of deep learning-based detection efficiency and selective extraction efficiency of cell aggregate-laden microcapsules;

FIG. 8D is a graph of exemplary data comparing the purity of microcapsules without and with the deep learning-based selective extraction;

FIG. 8E is a graph of exemplary data of cell viability in both control cell aggregates without microencapsulation or extraction and cell aggregates in microcapsules selectively extracted using the deep learning-enabled label-free method;

FIG. 9 is a flow chart of a method for selective microcapsule extraction according to aspects of the present disclosure;

FIG. 10A is an expanded flowchart of the system of FIG. 1;

FIG. 10B is a table listing exemplary microchannel dimensions of the system of FIG. 10A;

FIG. 11 is an illustration of an exemplary electric circuit connecting the electrodes, switch, and controller of the system of FIG. 10A;

FIG. 12 is a table of bright field and fluorescence images showing live/dead staining in both cell aggregates without going through microencapsulation or extraction (control) and cell aggregates in microcapsules selectively extracted using the deep learning-based label-free method;

FIG. 13 is a flow chart of a method for selective microcapsule extraction according to aspects of the present disclosure; and

FIG. 14 is a block diagram of an exemplary computer for implementing the method for selective microcapsule extraction according to aspects of the present disclosure.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

The presently disclosed subject matter relates generally to a system and method for detection and selective extraction of microcapsules. In certain embodiments, the system and method include machine learning-based detection. In certain embodiments, the system operates on a microfluidic chip. In an example embodiment, the method includes a highly efficient machine learning based label-free on-chip detection and selective extraction method for obtaining highly pure samples of cell/tissue-laden hydrogel microcapsules.

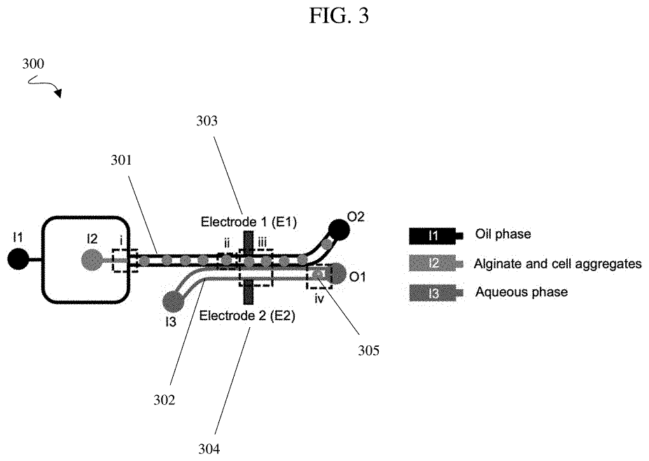

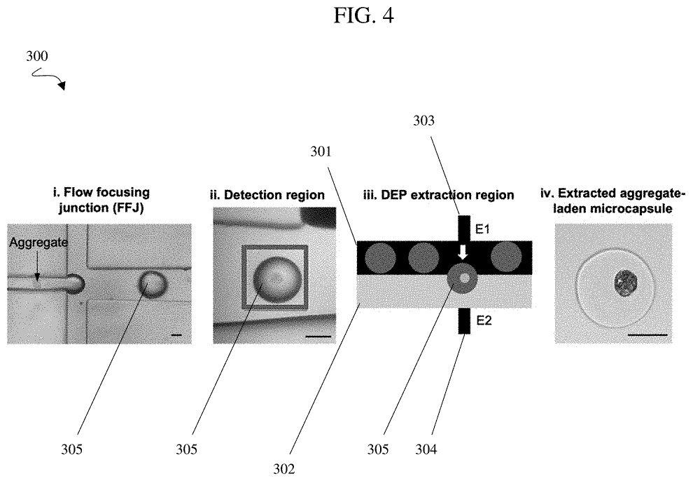

In the present disclosure, devices, systems, and methods for a highly efficient deep learning-enabled label-free on-chip detection and selective extraction of cell aggregate-laden hydrogel microcapsules are described. This is achieved through using categorically labeled images to train a deep learning-based detection model, which is then used to dynamically analyze the real-time images for label-free detection of the cell aggregate-laden microcapsules with ˜100% efficiency. Once detected, a DEP force is activated to extract the cell-laden microcapsules from oil into an aqueous phase with high efficiency (˜97%), high purity (˜90%), and high cell viability (>95%). DEP is a simple and fast method of moving particles that allows for a quick transfer of microcapsules from oil into an aqueous phase. An exemplary system includes a microfluidic device for microcapsule generation, a cell phone camera for imaging the on-chip detection area, a deep learning model for detection via analyzing the video frames from the camera in real-time, and a microcontroller that receives the output from the deep learning model and controls the switch to activate/deactivate the DEP-based extraction. In the microfluidic device (see, e.g., microfluidic device 300 in FIGS. 3 and 4), an aqueous solution of sodium alginate containing cell aggregates is connected to inlet 1 (I1), the oil phase that contains CaCl2 for gelling the sodium alginate solution into calcium alginate hydrogel is connected to I2, and the isotonic aqueous extraction solution is connected to I3. The flow rates are 200 μL/h, 2 mL/h, and 6 mL/h for the sodium alginate solution, oil phase, and extraction solution, respectively. At the flow focusing junction (FFJ, i), the aqueous alginate solution is sheared by the oil phase to form microdroplets. The microdroplets are gelled into microcapsules by CaCl2 in the oil phase at the FFJ and the downstream gelling microchannels before detection. Some of the microcapsules contain a cell aggregate and the majority of them are empty. Microcapsules may be identified containing aggregates, tissues, and/or at least one cell. Individual cells, groups of multiple cells, or cell aggregates may be encapsulated in a single microcapsule and identified.

The microcapsules further flow into the detection region (ii) where images are taken by a cell phone camera via the objective of a low-cost Zeiss (Oberkochen, Germany) Primovert brightfield light microscope. This can be done by attaching an iPhone® cell phone (Apple Inc., CA, USA) to the microscope with the phone camera being overlayed on the microscope eyepiece. The phone also relays the images to a computer. The deep learning model on the computer analyzes the input images in real-time to determine if the microcapsule currently in the detection region contains a cell aggregate or is empty. This information is then sent to a microcontroller, which controls a switch that turns on when the model determines there is a microcapsule containing a cell aggregate in the detection region. Based on the flow speed of the oil phase, distance between two adjacent microcapsules, inference time of the detection system, and time needed for DEP activation, a distance of 10 μm can be employed between the detection region and electrode location to ensure timely extraction of a detected cell aggregate-laden microcapsules with minimal interference of neighboring microcapsules. When the switch is turned on, an electric field is applied across the microchannel via the two electrodes (E1 and E2, located at 10 μm downstream of the detection region) to generate DEP force for selectively extracting the cell aggregate-laden microcapsule from the oil phase into the isotonic aqueous extraction solution (iii). The extracted microcapsules then flow to the outlet 1 (O1, iv), while non-extracted microcapsules stay in the oil phase and flow to the outlet O2. The microcapsules have a diameter of 219.4+/−8.2

A deep learning model can be utilized to enable label-free detection of cell aggregate-laden microcapsules in real-time. This is achieved through training the deep learning neural network model using pre-labeled (i.e., with or without a cell-laden microcapsule) images of the detection region. Once the model is trained, images from the cell phone camera showing the detection region of the microfluidic chip are read into the detection program and the model determines whether or not there is an aggregate-laden microcapsule in the detection region in real-time.

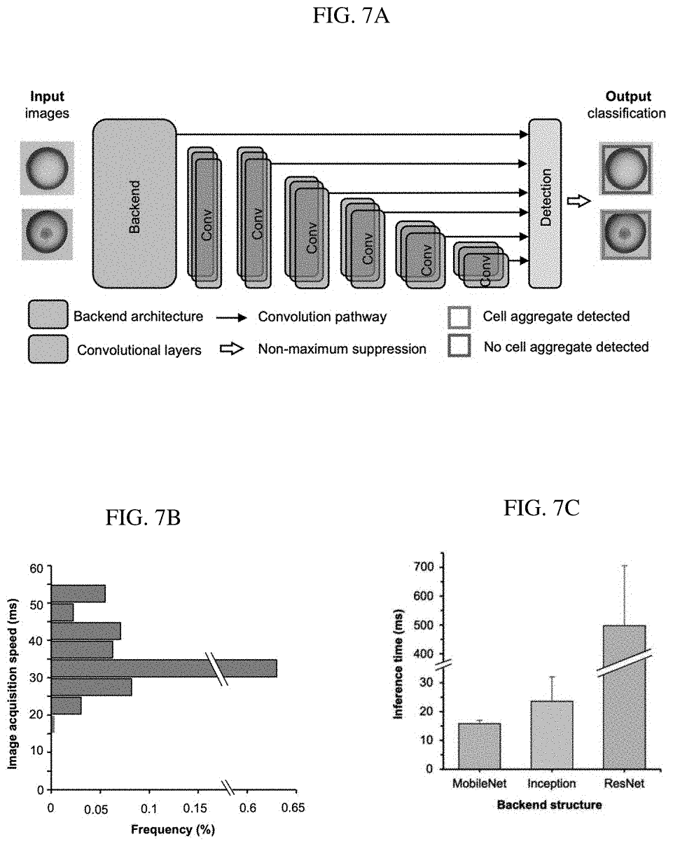

An exemplary detection model is based on the single shot multibox detector (SSD), a current state-of-the-art model for object detection. The detection model includes two components: a backend feature extractor, followed by several convolutional layers for bounding box predictions. The predicted bounding boxes are refined through non-maximum suppression. A comparison between three different backend structures is described (see, e.g., FIGS. 7A-7C) including MobileNet®, GoogLeNet® (Inception), and Residual-Net® (ResNet). The backend models are first pre-trained on the COCO dataset, an open source dataset of labeled images used for model training. Based on the speed needed for real-time detection and the imaging rate of the iPhone® camera (e.g., iPhone® 7), an acquisition speed of 30 frames per second (fps) can be employed, meaning the time interval between two adjacent image frames should be ˜33 ms. There may be some variability in the actual acquisition speed. The actual time interval between two adjacent image frames ranges from 18-50 ms. Therefore, for real-time application at 30 fps, the inference time (the time it takes the model to distinguish between an aggregate-laden and empty microcapsule in real-time) must be no greater than 18 ms. Using MobileNet as the backend structure, the model achieves the shortest inference time of ˜16 ms, while the inference time of the Inception model fluctuates between ˜15 and ˜33 ms and the ResNet model has the longest inference time of ˜400 ms. Based on this data, MobileNet has the shortest inference time (16 ms). To train the model using MobileNet as the backend structure, images of microcapsules in the detection region are taken and labeled as being cell aggregate-laden or empty (e.g., 400 for each) as the input images. Images of noise and air bubbles are also included in training to improve detection efficiency. The model is trained for 100,000 steps, using TensorFlow® (Google) on a GTX 1050ti GPU with 16 GB memory.

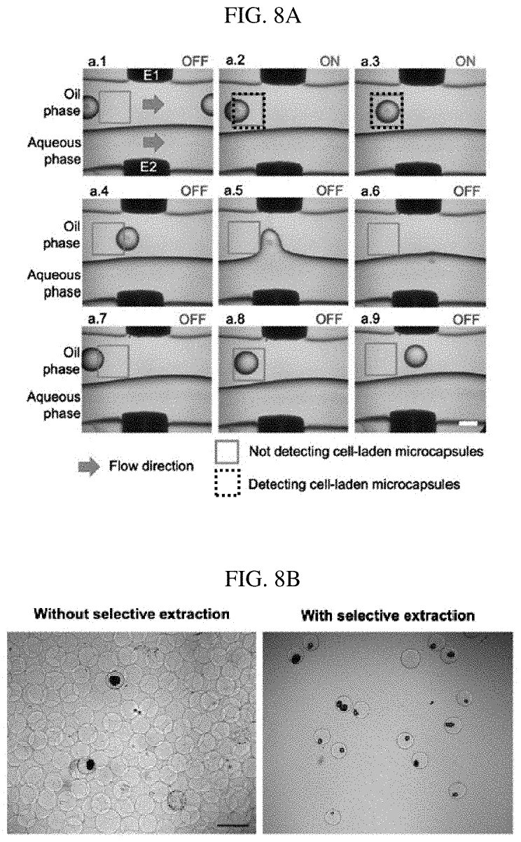

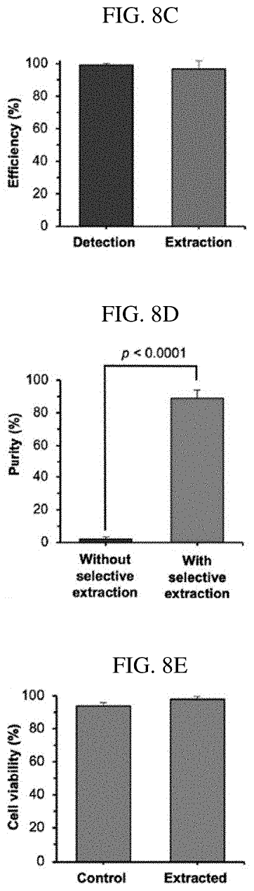

When real-time extraction is performed on cell aggregate-laden microcapsules, enabled by the deep learning-based label-free detection of the cell aggregate-laden microcapsules on-chip using the detection model trained with the MobileNet backend structure, the efficiency and purity of the approach described herein for both detection and selective extraction through microcapsule collection and counting, as well as quantification of cell viability is increased. A video frame breakdown of the deep learning-based detection and selective extraction is shown in FIG. 8A. After formation at the FFJ and further gelling in the downstream gelling microchannel, the microcapsule flows into the detection area. The deep learning detection model running on the computer can then determine if the microcapsule is empty or cell-laden. If a cell aggregate-laden microcapsule appears in the detection region, the computer program sends a corresponding signal to the microcontroller which may be connected to the computer through a USB (e.g., COME) connection. The microcontroller then activates DEP for 0.15 seconds to extract the cell aggregate-laden microcapsule from the oil phase into the aqueous phase). The duration of 0.15 seconds for DEP activation can be determined to achieve the extraction of one microcapsule per DEP activation, based on the speed of the microcapsules, time between microcapsules passing the extraction region, and time of extraction. If no cell aggregate-laden microcapsule is detected, DEP stays off and the microcapsule continues to flow down the oil channel.

The deep learning-based label-free methods described herein can detect cell aggregates (50-250 μm in diameter) with an ˜100% detection efficiency. To determine the selective extraction efficiency and purity as well as cell viability, microcapsules are collected from both the aqueous outlet O1 and oil outlet O2. The extraction efficiency is defined as the percent of extracted cell aggregate laden microcapsules out of all cell aggregate-ladened microcapsules, while the extraction purity is defined as the percent of the extracted cell aggregate-laden microcapsules out of the total extracted microcapsules. The purity of the selectively extracted microcapsules is significantly much higher than that (˜2%) without selective extraction.

The deep learning-based label-free method can detect cell aggregates of 50-250 μm in diameter with an ˜100% detection efficiency, which enables selective extraction of the cell aggregate laden microcapsules via DEP force with an ˜97% extraction efficiency. This high detection efficiency can also be attributed to the size of the microcapsules and the design of the devices and systems described herein, to ensure the cell aggregates are not far away from the plane of focus (i.e., within the depth of focus) so that they show up in the images used by the deep-learning algorithm for detection. This detection method is much better than a previously reported optical sensor-based approach that is unable to detect or extract any cell aggregates less than 82 μm. This is important for biomedical applications, for instance for islet microencapsulation, because islets can be as small as 50 μm. The purity (˜90%) of the deep learning-based extraction is also much higher than that (˜30%) achieved with an optical sensor-based detection method for DEP-based extraction.

The throughput of the devices, systems, and methods described herein is ˜1.5 microcapsules per second. This is due to the flow rates of the aqueous and oil phases used, which are optimized based on the time needed for detection and extraction, as well as the time needed for the oil/aqueous interface to become stable after extracting a cell-laden microcapsule. If the rate of microcapsule generation is too high and the microcapsules are too close to each other, the purity of the extracted cell-laden microcapsules may decrease. This is because the neighboring microcapsules (cell-laden or not) may be extracted either before the DEP is de-activated, or before the interface becomes stable after extracting a target cell-laden microcapsule. This may contribute partially to the low purity (˜30%) of conventional optical sensor-based systems with a throughput of ˜3.75 microcapsules per second. Nonetheless, for the applications of microencapsulating islets and follicles, usually ˜100 follicles or 1000 islets are needed at a time, making this throughput of ˜1.5 microcapsules per second sufficient. For applications that require higher throughput, smaller microcapsules that may cause less interface destabilization could be used, along with more viscous oil phase and aqueous extraction solution to further stabilize the interface, for increasing the throughput while keeping a high purity. The electrical conductivity of the oil phase can also be adjusted for faster extraction. Advances in deep learning and improved backend structures, along with a high-speed camera for imagining, and a faster computer processor and microcontroller can also increase throughput. Throughput may also be increased by running multiple microfluidic devices in parallel.

The deep learning system can also be trained with images of smaller aggregates (e.g., smaller than 50-250 μm for the application of microencapsulating single pancreatic islets and ovarian follicles that have a diameter over the same range) or single cells. The microfluidic device and detection system can also be adjusted to ensure that smaller aggregates and single cells can be identified and are within the depth of focus for imaging so that a high detection and extraction efficiency could be achieved. Reducing the microcapsule size may also help to increase detection efficiency, especially for detecting smaller aggregates or one single cell in the microcapsules. The position of cells, the plane of focus, and microcapsule edge opacity may vary with microcapsule size and can affect the detection efficiency. This type of model can also be applied to sort cell aggregates based on their size by training the model with images of aggregates of varying sizes in the microcapsules.

The elimination of cell labeling with the label-free devices, systems and methods described herein is of great significance to the use of the cells for downstream biomedical applications where labeled cells cannot be used, including treating type 1 diabetes with microencapsulated islets, as well as microencapsulation of an ovarian follicle for biomimetic 3D culture to treat infertility. The devices, systems, and methods described herein allow for a quick transfer of microcapsules from the time of detection to extraction (˜15 ms), and microcapsules are moved from the oil phase that is not favorable for the survival of living cells, to an aqueous solution in less than 10 seconds (determined by dividing the gelling microchannel length with the flow speed in the channel), from the time when the microcapsules are generated to their extraction. This contributes to the high cell viability of the extracted sample. This approach eliminates the need for tedious manual sorting of non-labeled aggregates and the associated possibility of sample contamination.

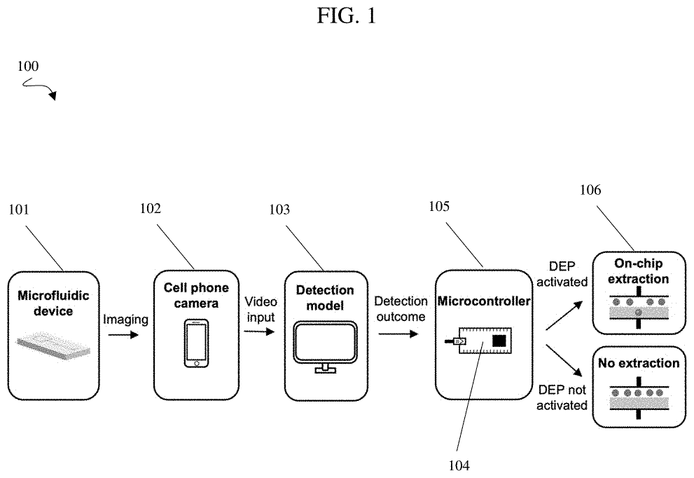

Referring generally to FIGS. 1-4, a deep-learning based detection and extraction device, system and method is described.

Referring particularly to FIG. 1, a deep-learning based detection and extraction system generally includes a microfluidic device 101, camera 102 , detection module 103, and a force generator 104 controlled by microcontroller 105. A force (e.g., dielectrophoresis) may be selectively applied to microcapsules for on-chip extraction 106 of microcapsules identified by a deep-learning based detection model as including aggregates, tissues, or at least one cell in a particular microcapsule.

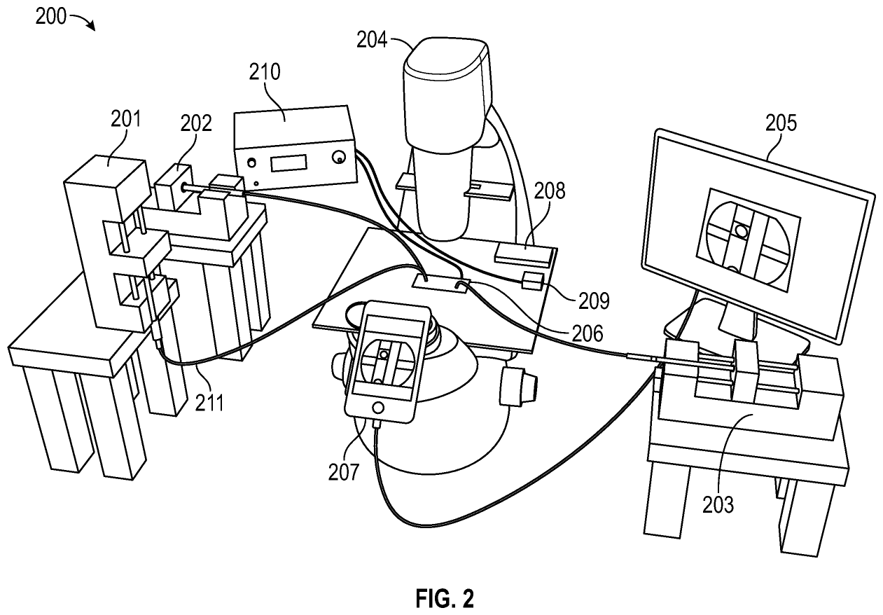

Referring particularly to FIG. 2 a system 200 for selective microcapsule extraction includes a first pump 201, a second pump 202, and a third pump 203 in fluid communication with the microfluidic device 206 (e.g., via a series of connection tubes 211). The first pump 201 is a vertical pump holding cells and the second and third pumps 202, 203 are horizontal pumps. A microscope 204 is employed for visualization of microcapsules passing through the microfluidic device 206. A computer 205 (see, e.g., computer 1400 described in more detail below) includes a processor and a memory and operates as the detection module (see, e.g., detection module 103 in FIG. 1). A camera 207 (e.g., a cell phone camera) is employed to capture images of the microcapsules in the microfluidic device 206. A microcontroller 208 controls a switch 209 that selectively activates a force generator (e.g., voltage generators 210) to apply a force to the microcapsules in the microfluidic device 206 to extract the microcapsules.

Referring particularly to FIG. 3, microfluidic device 300 includes a first phase 301 (e.g., an oil phase) and a second phase 302 (e.g., an aqueous phase). The first and second phases 301, 302 are immiscible phases. Microcapsules 305 can be extracted from the first phase 301 to the second phase 302 by selectively activating electrodes 303, 304 to apply a force, such as an electric force (e.g., dielectrophoresis), and acoustic force, or a mechanical force to microcapsules including aggregates, tissues, or at least one cell. FIG. 4. is an enlarged view of the steps performed by the microfluidic device 300.

The microfluidic device (see, e.g., microfluidic device 300 in FIGS. 3 and 4) includes an oil channel inlet 1 (I1) for flowing an oil phase (e.g., an oil emulsion containing CaCl2), an I2 for flowing isotonic aqueous sodium alginate solution suspended with cell aggregates, and an I3 for flowing the isotonic aqueous extraction solution. Microcapsules are formed at the flow focusing junction (FFJ, i in FIG. 4), gelled in the downstream gelling channel, and further flow into the detection region (ii in FIG. 4) where images are taken for real-time detection.

A deep learning-based detection program processes images of the microcapsules received from a camera (e.g., camera 102) to determine if the microcapsules contain a cell aggregate or are empty. Once a cell aggregate-laden microcapsule is detected, the microcontroller (e.g., microcontroller 105) is informed to turn the switch (e.g., switch 209) on, activating a force (e.g., a DEP force) to extract the cell aggregate-laden microcapsule into the aqueous extraction channel (iii in FIG. 4). Extracted microcapsules then flow down to the aqueous outlet (O1) where they are collected (iv in FIG. 4). Non-extracted microcapsules continue to flow with oil to outlet O2. (Scale bars: 100 μm).

Referring particularly to FIGS. 1, 5, 6A, and 6B the system 100 for selective microcapsule extraction includes a non-planar core-shell microfluidic device 101. The non-planar core-shell microfluidic device 101 generates microcapsules defining a core-shell configuration. A subset of the microcapsules contain aggregates, tissues, or at least one cell. The subset of microcapsules may encapsulate a single cell, multiple cells, or an aggregate of multiple cells. The machine learning models described herein can quantitatively identify a number of cells encapsulated in each microcapsule.

A camera 102 captures images of the microcapsules. The images may be digital images and may include video images. The camera 102 may be a digital camera, a high speed digital camera, a camera embodied in a smartphone, or a camera embodied in a tablet computer.

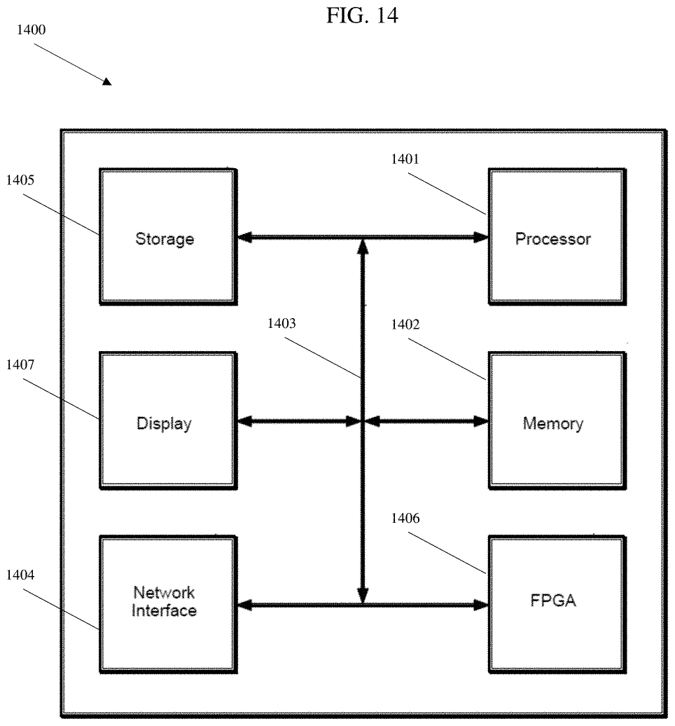

A detection module 103 includes a processor and a memory. The memory includes instructions that when executed by the processor cause the detection module 103 to provide the images of the microcapsules as an input to a machine learning model. The detection module may run on any computer, as described herein (see, e.g., the computer 1400 described in more detail below with reference to FIG. 14, and/or any specialized machine or computing device).

The machine learning model identifies microcapsules containing aggregates, tissues, or at least one cell. The machine learning model may include a machine learning classifier, or a convolutional neural network (see, e.g., FIGS. 7A, 7B and 7C). Other machine learning classifiers may be similarly employed for identifying microcapsules containing aggregates, tissues, or at least one cell.

A force generator 104 generates a force to extract the microcapsules. A microcontroller 105 selectively activates the force generator 104 to generate the force when the detection module 103 identifies a microcapsule containing aggregates, tissues, or at least one cell to extract the microcapsule. The force may be a non-invasive force.

In an aspect of the present disclosure, the force generated by the force generator 104 includes an electrical force, an acoustic force, or a mechanical force.

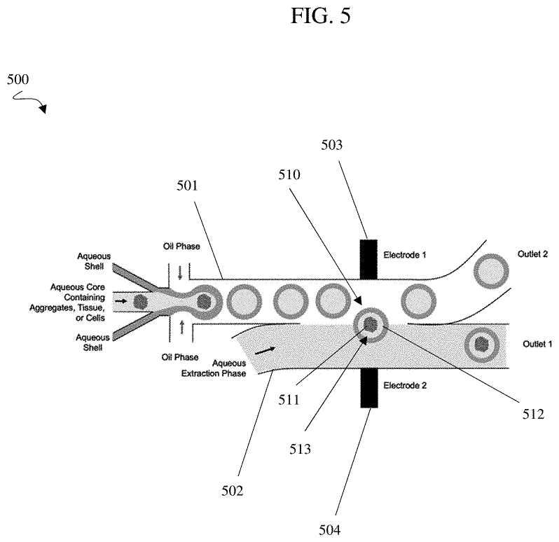

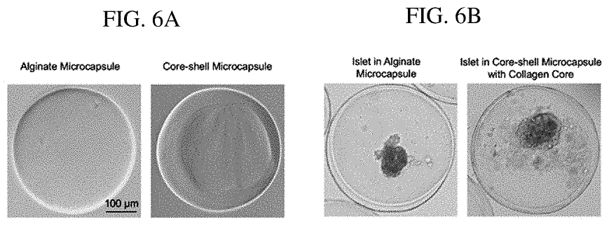

Referring particularly to FIGS. 5, 6A, and 6B, the non-planar core-shell microfluidic device 500 creates a core-shell microcapsule 510, which has a distinct core in the center of the microcapsules (see, e.g., FIG. 6A) in which different hydrogel materials can be used to create a biomimetic environment surrounding the cell(s), tissue(s), and/or aggregate(s) (see, e.g., FIG. 6B)). As opposed to a device having a planar design (i.e., channels that are all the same height), a non-planar core-shell microfluidic device has channels of varying heights (i.e., non-planar) which is essential for the creation of core-shell microcapsules (i.e., microcapsules having a core-shell configuration). The core-shell configuration ensures that the cell(s), tissue(s) or aggregate(s) are centered in the capsule, preventing detrimental effects of cell(s), tissue(s) or aggregate(s) being partially exposed. For example, an islet that is partially exposed from a microcapsule can greatly increase immune response during an islet transplantation, and the core-shell configuration ensures an alginate barrier between the islet and the microcapsule edge.

Referring particularly to FIG. 5, a core 511 of a microcapsule 510 generated by the non-planar core-shell microfluidic device 500 is surrounded by an inner wall 512 of the microcapsule 510. The core 511 may be centered in an inner space 513 of the microcapsule 510.

In an aspect of the present disclosure, the microcapsule 510 generated by the non-planar core-shell microfluidic device 500 defines a biomimetic environment containing a plurality of single cells, a plurality of cell tissues, and a plurality of cell aggregates. The biomimetic environment may also contain a single cell. The biomimetic environment may include at least one hydrogel surrounding the aggregates, tissues, or cell(s).

The microfluidic device 500 includes at least two immiscible phases (e.g., an oil phase 501 and an aqueous phase 502). The force generator is selectively activated by the microcontroller to extract microcapsules from a first phase to a second phase (e.g., by activating electrodes 503, 504).

Referring particularly to FIGS. 6A and 6B, a conventional alginate microcapsule is compared with a core-shell microcapsule generated by the non-planar core-shell microfluidic device 500. The alginate microcapsule has a solid alginate microcapsule and the core-shell microcapsule allows for the use of different core materials. FIG. 6B illustrates an example of an islet encapsulated in an alginate microcapsule (left) and a core-shell microcapsule with a biomimetic collagen-based core and an alginate shell (right).

In an aspect of the present disclosure, the extracted microcapsule 510 includes ovarian follicle cells, pancreatic islet cells, stem cells, or somatic cells. The ovarian follicle cells, pancreatic islet cells, stem cells, or somatic cells are arranged as a single cell, multiple cells, or cell aggregates.

Referring to FIGS. 7A, 7B and 7C, the deep learning detection model is described in more detail. As an example, the deep learning detection model includes a backend architecture and a convolutional neural network using a single shot multibox detection (SSD), for detecting cell aggregate-laden microcapsules. FIG. 7B illustrates exemplary data showing the image acquisition speed using the iPhone® 7 camera. FIG. 7C illustrates exemplary inference times of the deep learning detection models with three different backend structures.

Referring to FIGS. 8A, 8B, 8C, 8D, and 8E, the deep learning-enabled detection and selective extraction of cell aggregate-laden microcapsules is illustrated. FIG. 8A illustrates an exemplary image sequence showing the selective extraction of a cell aggregate-laden microcapsule from oil into the aqueous extraction solution. FIG. 8B illustrates exemplary images of microcapsules collected without and with selective extractions, showing the difference in purity between the samples. (Scale bar: 500 μm). FIG. 8C illustrates exemplary data of the deep learning-based detection efficiency and selective extraction efficiency of cell aggregate-laden microcapsules (n=3 independent runs with ˜1000 microcapsules per run). FIG. 8D illustrates exemplary data comparing the purity of microcapsules without and with the deep learning-based selective extraction (n=3 independent runs with 1000 microcapsules per run for each condition). FIG. 8E illustrates exemplary data of cell viability in both control cell aggregates without microencapsulation or extraction and cell aggregates in microcapsules selectively extracted using the deep learning-enabled label-free method (n=3 independent runs with 7 cell aggregates per run for each condition).

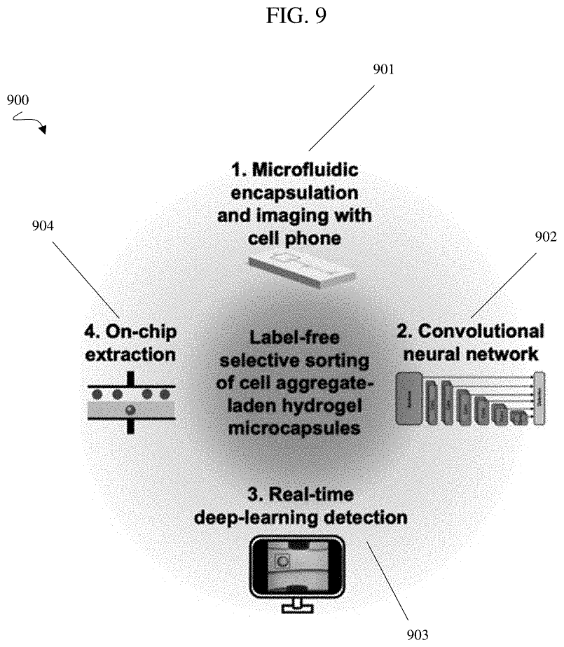

Referring to FIG. 9, a method 900 of label-free selective sorting of cell-aggregate laden hydrogel microcapsules includes encapsulation of aggregates, tissues, or at least one cell and imaging of the microcapsules using a camera (e.g., a cell phone camera) at step 901. A convolutional neural network is employed to identify the microcapsules including the aggregates, tissues and/or cell(s) at step 902 to achieve real-time deep-learning detection of the cell-laden microcapsules at step 903. On-chip extraction is performed at step 904 to extract the cell-laden microcapsules.

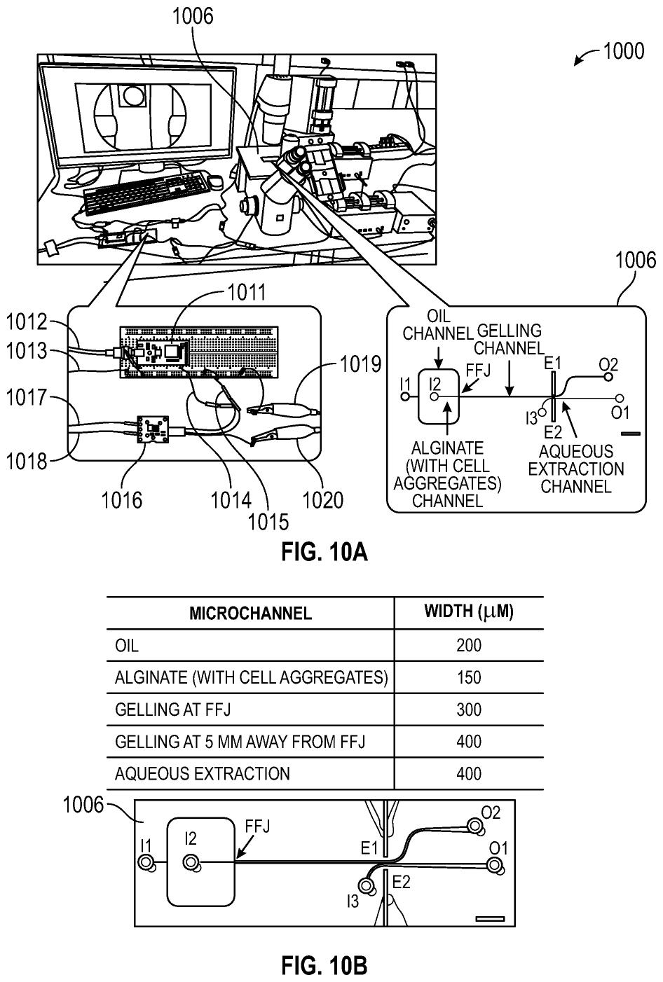

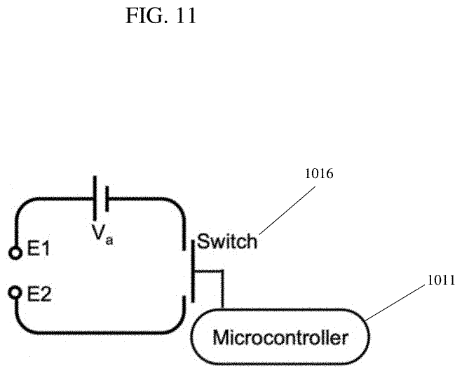

The system 1000 described below with reference to FIGS. 10A, 10B, and 11 is substantially the same as the system 200 described above, unless otherwise indicated below (see, e.g., microfluidic device 1006 and microcontroller 1008), and thus duplicative descriptions will be omitted below.

Referring to FIGS. 10A, 10B, and 11, a circuit connection between microcontroller 1011 and switch 1016 is described. The electric circuit connecting the electrodes (see, E1, E2), switch 1016, and microcontroller 1011 is illustrated in expanded form (Va: 250 V). The circuit connection includes a deep learning program output from the computer to the microcontroller 1012, a ground for electrode E2 and high voltage generator 1013, a microcontroller output (P18) to switch on/off the signal 1014 and a ground 1015 for switch 1016. The circuit connection also includes a voltage input from the high power generator to the switch 1017, a switch to electrode E1 for DEP voltage 1018, a ground for the switch power source 1019 and a switch power source 1020. Referring particularly to FIG. 10B, a table listing the microchannel dimensions of microfluidic device 1006 is provided. As an example, the gelling microchannel is 300 μm in width at the flow-focusing junction (FFJ) and it gradually increases to 400 μm at 5 mm away (downstream) from the FFJ. All channels are 250 μm in height. (Scale bars: 4 mm).

Referring particularly to FIG. 11, an electric circuit connecting electrodes (E1, E2), switch 1016 and microcontroller 1011 is illustrated.

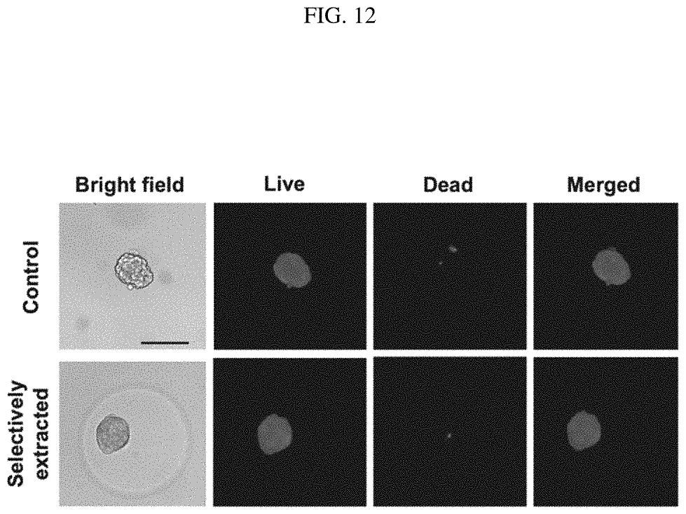

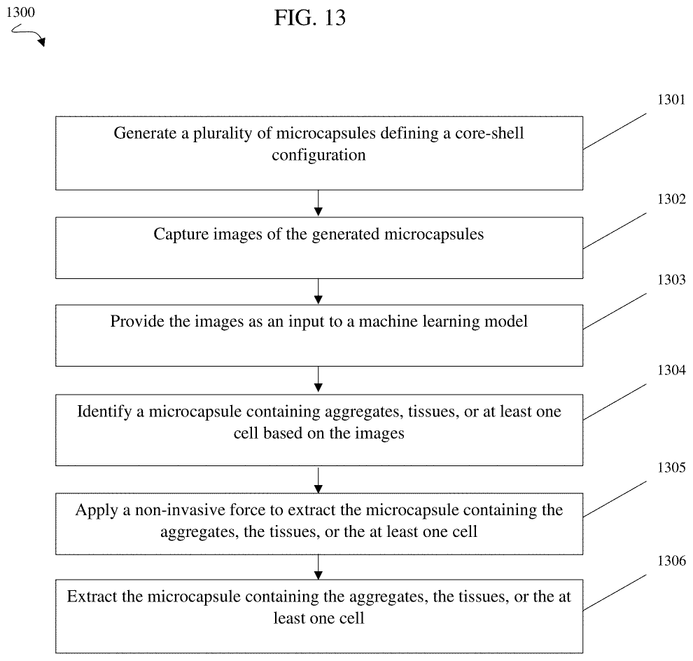

Referring to FIG. 12, characterization of cell viability in cell aggregates is illustrated. Bright field and fluorescence images showing live/dead staining in both cell aggregates without going through microencapsulation or extraction (control) and cell aggregates in microcapsules selectively extracted using the deep learning-based label-free method are illustrated. (Scale bar: 100 μm).

With ongoing reference to FIG. 12, the cell viability is examined by live/dead staining to determine if the DEP extraction process causes any damage to the cells, since cells have to be highly viable for their further biomedical applications. The cell aggregates in the microcapsules collected from the aqueous outlet O2 are labeled using live/dead (green/red) staining and imaged with fluorescence microscopy, and the green and red areas in the images are quantified. Typical fluorescence images are provided, which shows negligible dead stain, indicating the cells are highly viable. Quantitative analysis of the live/dead areas shows more than 95% of cells in the selectively extracted microcapsules (n=3 independent runs) are viable, which is not significantly different from that of fresh cells (control) in the cell aggregates (n=3 independent runs) without microencapsulation or extraction.

Referring to FIG. 13, a computer-implemented method 1300 for selective microcapsule extraction includes generating microcapsules defining a core-shell configuration 1301. A subset of the microcapsules contain aggregates, tissues, or at least one cell. Images of the generated microcapsules are captured 1302. The images are provided as an input to a machine learning model 1303. The machine learning model identifies a microcapsule containing aggregates, tissues, or at least one cell based on the provided input 1304. A non-invasive force is selectively applied 1305 to extract the microcapsule containing aggregates, tissues, or at least one cell to extract the microcapsule 1306.

In an aspect of the present disclosure, the extracted microcapsule is provided as an in-vivo treatment, for an in-vitro study, or for cell analysis.

Referring to FIG. 14, a computer 1400 is described. The computer 1400 can be employed to run the detection module, as described herein. The computer 1400 may include a processor 1401 connected to a computer-readable storage medium or a memory 1402 which may be a volatile type memory, e.g., RAM, or a non-volatile type memory, e.g., flash media, disk media, etc. The processor 1401 may be another type of processor such as, without limitation, a digital signal processor, a microprocessor, an ASIC, a graphics processing unit (GPU), field-programmable gate array (FPGA), or a central processing unit (CPU).

In some aspects of the disclosure, the memory 1402 can be random access memory, read-only memory, magnetic disk memory, solid-state memory, optical disc memory, and/or another type of memory. The memory 1402 can communicate with the processor 1401 through communication buses 1403 of a circuit board and/or through communication cables such as serial ATA cables or other types of cables. The memory 1402 includes computer-readable instructions that are executable by the processor 1401 to operate the computer 1400 to execute the algorithms described herein. The computer 1400 may include a network interface 1404 to communicate (e.g., through a wired or wireless connection) with other computers or a server. A storage device 1405 may be used for storing data. The computer 1400 may include one or more FPGAs 1406. The FPGA 1406 may be used for executing various machine learning algorithms. A display 1407 may be employed to display data processed by the computer 1400.

The machine learning models described herein (e.g., including a neural network) can be trained using a dataset including known matching and non-matching entries (e.g., data of previously verified microcapsules including aggregates, tissues, or at least one cell and verified microcapsules not including any cells, aggregates or tissues). The machine learning models may be trained on datasets for multiple cells or aggregates of cells contained in a core-shell microcapsule, as described herein.

An exemplary training process for the deep-learning model is described in more detail below. The deep learning model can be trained using labeled images of empty and cell aggregate-laden microcapsules. Images of air bubbles and noise in the oil phase are included to help the model distinguish between noise, microcapsules, and cell aggregates. First, iVcam is used to record videos of microcapsules in the detection region using an iPhone® attached to the eyepiece of a Zeiss Primovert microscope. Then the videos are split into frames, and frames with empty microcapsules and cell aggregate-laden microcapsules are collected (400 empty and 400 aggregate-laden). Images are cropped to include only one microcapsule and labeled as “empty” or “cell aggregate-laden” using the program LabelImg in the Python® Package Index (PyPI). Labeled image data is then divided randomly into training and testing data (80% training, 20% testing). Images are used to train the deep learning models using Tensorflow® (Google) for 100,000 steps. Testing data is then used to confirm the model's detection precision.

The documents listed below and referenced herein are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

J. Zhang, R. J. Coulston, S. T. Jones, J. Geng, O. A. Scherman, C. Abell, Science (80-.). 2012, 335, 690.

A. S. Mao, B. Ozkale, N. J. Shah, K. H. Vining, T. Descombes, L. Zhang, C. M. Tringides, S. W. Wong, J. W. Shin, D. T. Scadden, D. A. Weitz, D. J. Mooney, Proc. Natl. Acad. Sci. U.S.A. 2019, 116, 15392.

A. J. Vegas, O. Veiseh, M. Gürtler, J. R. Millman, F. W. Pagliuca, A. R. Bader, J. C. Doloff, J. Li, M. Chen, K. Olejnik, H. H. Tam, S. Jhunjhunwala, E. Langan, S. Aresta- Dasilva, S. Gandham, J. J. McGarrigle, M. A. Bochenek, J. Hollister-Lock, J. Oberholzer, D. L. Greiner, G. C. Weir, D. A. Melton, R. Langer, D. G. Anderson, Nat. Med. 2016, 22, 306.

X. He, ACS Biomater. Sci. Eng. 2017, 3, 2692.

W. Zhang, X. He, J. Healthc. Eng. 2011, 2, 427.

P. Agarwal, J. K. Choi, H. Huang, S. Zhao, J. Dumbleton, J. Li, X. He, Part. Part. Syst. Charact. 2015, 32, 809.

J. K. Choi, P. Agarwal, H. Huang, S. Zhao, X. He, Biomaterials 2014, 35, 5122.

M. Ma, A. Chiu, G. Sahay, J. C. Doloff, N. Dholakia, R. Thakrar, J. Cohen, A. Vegas, D. Chen, K. M. Bratlie, T. Dang, R. L. York, J. Hollister-Lock, G. C. Weir, D. G. Anderson, Adv. Healthc. Mater. 2013, 2, 667.

A. M. White, J. G. Shamul, J. Xu, S. Stewart, J. S. Bromberg, X. He, ACS Biomater. Sci. Eng. 2020, 6, 2543.

S. Zhao, Z. Xu, H. Wang, B. E. Reese, L. V Gushchina, M. Jiang, P. Agarwal, J. Xu, M. Zhang, R. Shen, Z. Liu, N. Weisleder, X. He, Nat. Commun. 2016, 7, 1.

R. Seemann, M. Brinkmann, T. Pfohl, S. Herminghaus, Reports Prog. Phys. 2012, 75, 016601.

D. M. Headen, G. Aubry, H. Lu, A. J. Garcia, Adv. Mater. 2014, 26, 3003.

L. Shang, Y. Cheng, Y. Zhao, Chem. Rev. 2017, 117, 7964.

H. Huang, X. He, Lab Chip 2015, 15, 4197.

K. Y. Lee, D. J. Mooney, Prog. Polym. Sci. 2012, 37, 106.

D. J. Collins, A. Neild, A. DeMello, A. Q. Liu, Y. Ai, Lab Chip 2015, 15, 3439.

M. De Groot, B. J. De Haan, P. P. M. Keizer, T. A. Schuurs, R. Van Schilfgaarde, H. G. D. Leuvenink, Lab. Anim. 2004, 38, 200.

D. Dufrane, W. D'hoore, R. M. Goebbels, A. Saliez, Y. Guiot, P. Gianello,

Xenotransplantation 2006, 13, 204.

X. He, T. L. Toth, Semin. Cell Dev. Biol. 2017, 61, 140.

M. Sun, P. Durkin, J. Li, T. L. Toth, X. He, ACS Sensors 2018, 3, 410.

H. Wang, P. Agarwal, B. Jiang, S. Stewart, X. Liu, Y. Liang, B. Hancioglu, A. Webb, J. P. Fisher, Z. Liu, X. Lu, K. H. R. Tkaczuk, X. He, Adv. Sci. 2020, 7, 2000259.

H. Huang, M. Sun, T. Heisler-Taylor, A. Kiourti, J. Volakis, G. Lafyatis, X. He, Small 2015, 11, 5369.

J. Nam, H. Lim, C. Kim, J. Yoon Kang, S. Shin, Biomicrofluidics 2012, 6, 024120.

A. Sciambi, A. R. Abate, Lab Chip 2015, 15, 47.

X. He, Ann. Biomed. Eng. 2017, 45, 1676.

P. R. O'Neill, W. K. A. Karunarathne, V. Kalyanaraman, J. R. Silvius, N. Gautama, Proc. Natl. Acad. Sci. U.S.A. 2012, 109, 20784.

D. R. Gossett, H. T. K. Tse, J. S. Dudani, K. Goda, T. A. Woods, S. W. Graves, D. Di Carlo, Small 2012, 8, 2757.

E. Pariset, C. Pudda, F. Boizot, N. Verplanck, J. Berthier, A. Thuaire, V. Agache, Small 2017, 13, DOI 10.1002/sm11.201770201.

T. S. H. Tran, B. D. Ho, J. P. Beech, J. 0. Tegenfeldt, Lab Chip 2017, 17, 3592.

E. H. M. Wong, E. Rondeau, P. Schuetz, J. Cooper-White, Lab Chip 2009, 9, 2582.

J. J. Agresti, E. Antipov, A. R. Abate, K. Ahn, A. C. Rowat, J. C. Baret, M. Marquez, A. M. Klibanov, A. D. Griffiths, D. A. Weitz, Proc. Natl. Acad. Sci. U.S.A. 2010, 107, 4004.

Z. Cao, F. Chen, N. Bao, H. He, P. Xu, S. Jana, S. Jung, H. Lian, C. Lu, Lab Chip 2013, 13, 171.

S. Webb, Nature 2018, 554, 555.

X. Lin, Y. Rivenson, N. T. Yardimci, M. Veli, Y. Luo, M. Jarrahi, A. Ozcan, Science (80-.). 2018, 361, 1004.

E. M. Christiansen, S. J. Yang, D. M. Ando, A. Javaherian, G. Skibinski, S. Lipnick, E. Mount, A. O'Neil, K. Shah, A. K. Lee, P. Goyal, W. Fedus, R. Poplin, A. Esteva, M. Berndl, L. L. Rubin, P. Nelson, S. Finkbeiner, Cell 2018, 173, 792.

Y. Wu, H. Shroff, Nat. Methods 2018, 15, 1011.

P. Zhang, S. Liu, A. Chaurasia, D. Ma, M. J. Mlodzianoski, E. Culurciello, F. Huang, Nat. Methods 2018, 15, 913.

A. S. Adamson, A. Smith, JAMA Dermatology 2018, 154, 1247.

T. Y. Lin, M. Maire, S. Belongie, J. Hays, P. Perona, D. Ramanan, P. Dollar, C. L. Zitnick, in Lect. Notes Comput. Sci. (Including Subser. Lect. Notes Artif. Intell. Lect. Notes Bioinformatics), 2014, pp. 740-755.

J. Schmidhuber, Neural Networks 2015, 61, 85.

Y. LeCun, Y. Bengio, G. Hinton, Nature 2015, 521, 436.

Y. J. Heo, D. Lee, J. Kang, K. Lee, W. K. Chung, Sci. Rep. 2017, 7, 1.

Z. Zhang, J. Ge, Z. Gong, J. Chen, C. Wang, Y. Sun, Int. J. Lab. Hematol. 2020, DOI 10.1111/ijlh.13380.

V. Anagnostidis, B. Sherlock, J. Metz, P. Mair, F. Hollfelder, F. Gielen, Lab Chip 2020, 20, 889.

M. Girault, H. Kim, H. Arakawa, K. Matsuura, M. Odaka, A. Hattori, H. Terazono, K. Yasuda, Sci. Rep. 2017, 7, DOI 10.1038/srep40072.

A. Chu, D. Nguyen, S. S. Talathi, A. C. Wilson, C. Ye, W. L. Smith, A. D. Kaplan, E. B. Duoss, J. K. Stolaroff, B. Giera, Lab Chip 2019, 19, 1808.

J. Huang, V. Rathod, C. Sun, M. Zhu, A. Korattikara, A. Fathi, I. Fischer, Z. Wojna, Y. Song, S. Guadarrama, K. Murphy, in Proc.—30th IEEE Conf. Comput. Vis. Pattern Recognition, CVPR 2017, 2017, pp. 3296-3305.

W. Liu, D. Anguelov, D. Erhan, C. Szegedy, S. Reed, C. Y. Fu, A. C. Berg, in Lect. Notes Comput. Sci. (Including Subser. Lect. Notes Artif. Intell. Lect. Notes Bioinformatics), 2016, pp. 21-37.

A. Mousavian, D. Anguelov, J. Košecká, J. Flynn, in Proc.—30th IEEE Conf. Comput.

Vis. Pattern Recognition, CVPR 2017, 2017, pp. 5632-5640.

S. Ren, K. He, R. Girshick, J. Sun, in Adv. Neural Inf. Process. Syst., 2015, pp. 91-99.

A. Neubeck, L. Van Gool, in Proc. - Int. Conf. Pattern Recognit., 2006, pp. 850-855.

A. G. Howard, M. Zhu, B. Chen, D. Kalenichenko, W. Wang, T. Weyand, M. Andreetto, H. Adam, arXiv 2017, 1704.04861.

C. Szegedy, W. Liu, Y. Jia, P. Sermanet, S. Reed, D. Anguelov, D. Erhan, V. Vanhoucke, A. Rabinovich, in Proc. IEEE Comput. Soc. Conf. Comput. Vis. Pattern Recognit., 2015, pp. 1-9.

K. He, X. Zhang, S. Ren, J. Sun, in Proc. IEEE Comput. Soc. Conf. Comput. Vis. Pattern Recognit., 2016, pp. 770-778.

A. C. Schapiro, T. T. Rogers, N. I. Cordova, N. B. Turk-Browne, M. M. Botvinick, in ArXiv Prepr., 2016.

J. Jo, Y. C. Moo, D. S. Koh, Biophys. J. 2007, 93, 2655.

H. Huang, X. He, Appl. Phys. Lett. 2014, 105, 143704.

MicroChem, “SU-8 2000 (2000.5-2015) Permanent Epoxy Negative Photoresist PROCESSING GUIDELINES FOR: SU-8 2100 and SU-8 2150,” can be found under www.atgc.cajp, 2010.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A system for selective microcapsule extraction, comprising:
a non-planar core-shell microfluidic device configured to generate a plurality of microcapsules defining a core-shell configuration, wherein a subset of the microcapsules of the plurality of microcapsules contain aggregates, tissues, or at least one cell;
a camera configured to capture a plurality of images of the generated plurality of microcapsules;
a detection module including a processor and a memory, the memory including instructions stored thereon which when executed by the processor causes the detection module to:
provide the plurality of images of the generated plurality of microcapsules as an input to a machine learning model; and
identify, by the machine learning model, a microcapsule of the subset of microcapsules containing aggregates, tissues, or at least one cell;
a force generator configured to generate a non-invasive force to extract the microcapsule of the subset of microcapsules; and
a microcontroller configured to selectively activate the force generator to generate the non-invasive force when the detection module identifies the microcapsule of the subset of microcapsules to extract the microcapsule of the subset of microcapsules.

2. The system of claim 1, wherein at least one microcapsule of the subset of microcapsules generated by the non-planar core-shell microfluidic device defines a biomimetic environment containing a plurality of single cells, a plurality of cell tissues, and a plurality of cell aggregates.

3. The system of claim 2, wherein the biomimetic environment includes at least one hydrogel surrounding the plurality of single cells, the plurality of cell tissues, and the plurality of cell aggregates.

4. The system of claim 1, wherein the microfluidic device includes at least two immiscible phases, and wherein the force generator is selectively activated by the microcontroller to extract the microcapsule of the subset of microcapsules from a first phase of the at least two immiscible phases to a second phase of the at least two immiscible phases.

5. The system of claim 1, wherein a core of at least one microcapsule of the subset of microcapsules generated by the non-planar core-shell microfluidic device is surrounded by an inner wall of the at least one microcapsule of the subset of microcapsules.

6. The system of claim 5, wherein the core of the at least one microcapsule of the subset of microcapsules is substantially centered within an inner space of the corresponding microcapsule of the plurality of microcapsules.

7. The system of claim 1, wherein the machine learning model includes a machine learning classifier, or a convolutional neural network.

8. The system of claim 1, wherein the extracted microcapsule of the subset of microcapsules includes ovarian follicle cells, pancreatic islet cells, stem cells, or somatic cells, and wherein the ovarian follicle cells, pancreatic islet cells, stem cells, or somatic cells are arranged as a single cell, multiple cells, or cell aggregates.

9. The system of claim 1, wherein the non-invasive force generated by the force generator includes an electrical force, an acoustic force, or a mechanical force.

10. The system of claim 1, wherein the camera is a digital camera, a high speed digital camera, a camera embodied in a smartphone, or a camera embodied in a tablet computer.

11. A computer-implemented method for selective microcapsule extraction, comprising:
generating a plurality of microcapsules, wherein a subset of the microcapsules of the plurality of microcapsules contain aggregates, tissues, or at least one cell;
capturing a plurality of images of the generated plurality of microcapsules;
providing the plurality of images of the generated plurality of microcapsules as an input to a machine learning model;
identifying, by the machine learning model, a microcapsule of the subset of microcapsules containing aggregates, tissues, or at least one cell based on the provided input; and
selectively applying a non-invasive force to extract the microcapsule of the subset of microcapsules containing aggregates, tissues, or at least one cell.

12. The computer-implemented method of claim 11, wherein generating the plurality of microcapsules includes generating at least microcapsule defining a biomimetic environment containing a plurality of cells, a plurality of cell tissues, and a plurality of cell aggregates.

13. The computer-implemented method of claim 12, further including generating the biomimetic environment to include at least one hydrogel surrounding the plurality of cells, the plurality of cell tissues, and the plurality of cell aggregates.

14. The computer-implemented method of claim 11, wherein extracting the microcapsule of the subset of microcapsules includes extracting the microcapsule of the subset of microcapsules from a first phase to a second phase of at least two immiscible phases.

15. The computer-implemented method of claim 11, wherein generating the plurality of microcapsules includes generating at least one core surrounded by an inner wall of a corresponding microcapsule of the plurality of microcapsules.

16. The computer-implemented method of claim 11, wherein generating the plurality of microcapsules includes generating at least one core substantially centered within an inner space of a corresponding microcapsule of the plurality of microcapsules.

17. The computer-implemented method of claim 11, wherein the plurality of images of the generated plurality of microcapsules is provided as an input to a machine learning classifier, or a convolutional neural network.

18. The computer-implemented method of claim 11, wherein extracting the microcapsule of the subset of microcapsules includes extracting ovarian follicle cells, pancreatic islet cells, stem cells, or somatic cells, and wherein the ovarian follicle cells, pancreatic islet cells, stem cells, or somatic cells are arranged as a single cell, multiple cells, or cell aggregates.

19. The computer-implemented method of claim 11, further including delivering the extracted microcapsule of the subset of microcapsules as an in-vivo treatment, providing the extracted microcapsule of the subset of microcapsules for an in-vitro study, or providing the extracted microcapsule of the subset of microcapsules for cell analysis.

20. A system for selective microcapsule or droplet extraction, comprising:
a non-planar core-shell microfluidic device configured to generate a plurality of microcapsules or a plurality of droplets defining a core-shell configuration, wherein a subset of the microcapsules of the plurality of microcapsules or a subset of the droplets of the plurality of droplets contain aggregates, tissues, or at least one cell;
a camera configured to capture a plurality of images of the generated plurality of microcapsules or the generated plurality of droplets;
a detection module including a processor and a memory, the memory including instructions stored thereon which when executed by the processor causes the detection module to:
provide the plurality of images of the generated plurality of microcapsules or the generated plurality of droplets as an input to a machine learning model; and
identify, by the machine learning model, a microcapsule of the subset of microcapsules or a droplet of the subset of droplets containing aggregates, tissues, or at least one cell;
a force generator configured to generate a force to extract the microcapsule of the subset of microcapsules or the droplet of the subset of droplets; and
a microcontroller configured selectively activate the force generator to generate the force when the detection module identifies the microcapsule of the subset of microcapsules or the droplet of the subset of droplets to extract the microcapsule of the subset of microcapsules or the droplet of the subset of droplets.
